(19) United States
(12) Patent Application Publication
(10) Pub. No.: US 2007/0238992 A1
(43) Pub. Date: Oct. 11, 2007

(54) Implantable sensor

(75) Inventors: William T. Donofrio, Andover, MN (US); Jeffrey H. Nycz, Collierville, TN (US); Can Cinbis, Shoreview, MN (US); Matthew M. Morrison, Cordova, TN (US); Susan J. Drapeau, Cordova, TN (US); Qingshan (Sam) Ye, Plymouth, MN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/344,667

(22) Filed: Feb. 1, 2006

(51) Int. Cl. A61B 8/00

(52) U.S. Cl. 600/437

(57) ABSTRACT

An implantable sensor for detecting changes in tissue density is disclosed. The implantable sensor includes a transducer adapted for detecting indicators of tissue density. The implantable sensor includes memory for storing data corresponding to the tissue density indicators detected by the sensor. A telemetry circuit is configured for transmitting the tissue density data outside of the body.

### FIELD OF THE INVENTION

The present invention is directed to improved instrumentation and methods for measuring tissue density. More particularly, in one aspect the present invention is directed to an implantable sensor for detecting changes in tissue density.

### BACKGROUND OF THE INVENTION

The present invention relates to the assessment of tissue density. The invention may have particularly useful application in the assessment of tissue density as it relates to total joint replacement surgeries including the implantation of hip, knee, shoulder, ankle, spinal and wrist prostheses. The invention may also have particularly useful application in the assessment of tissue density as it relates to soft tissue repairs such as ACL reconstruction or meniscal reconstruction, for example.

Joint prostheses are usually manufactured of durable materials such as metals, ceramics, or hard plastics and are affixed to articulating ends of the bones of the joint. Joint prostheses usually include an articulating surface composed of a material designed to minimize the friction between the components of the joint prostheses. For example, in a hip prosthesis the femoral component is comprised of a head (or ball) and a stem attached to the femur. The acetabular component is comprised of a cup (or socket) attached to the acetabulum and most often includes a polyethylene articulating surface. The ball-in-socket motion between the femoral head and the acetabular cup simulates the natural motion of the hip joint and the polyethylene surface helps to minimize friction during articulation of the ball and socket.

Total joint surgery often requires implanting components that articulate against polyethylene or metal bearing surfaces. This articulation has been shown to release submicron particle wear debris, often polyethylene wear debris. This debris may lead to osteolytic lesions, implant loosing, and possibly the need for revision surgery. Early detection of particle wear debris or the onset of osteolytic lesions allows an orthopedic surgeon to treat the potential problem before it escalates to the point of causing severe medical harm to the patient or the need for revision surgery.

Further, in soft tissue repairs, such as ACL reconstruction, the tissue may have problems with graft incorporation or failure to fully heal the defect. Tracking the healing process and tissue integrity in soft tissue repairs can assist the surgeon in determining the appropriate postoperative treatments and physical therapy. Also, early detection of a potential problem provides the surgeon with the potential ability to treat the affected tissue before the problem becomes more serious or requires revision surgery.

Therefore, there remains a need for improved instrumentation and methods for measuring tissue density and changes in tissue density.

### SUMMARY OF THE INVENTION

The present invention provides an implantable sensor for detecting indicators of tissue density that comprises a sensing element adapted for placement in natural tissue and configured for detecting a signal indicative of a density of a monitored tissue and a telemetry circuit in communication with the sensing element adapted for transmitting the detected signal outside of the natural tissue.

In another aspect, the present invention provides a system for detecting changes in tissue density that comprises an implantable acoustic sensor adapted for detecting a signal indicative of a density of a tissue and communicating the signal to an external receiver and an external receiver adapted for receiving the signal from the implantable sensor.

In another aspect, the present invention provides a method of evaluating the density of a tissue in a body that comprises implanting a sensor into natural tissue of the body, the sensor adapted for detecting a signal indicative of the density of the tissue, obtaining the detected signal from the sensor, and analyzing the signal to evaluate tissue density.

Further aspects, forms, embodiments, objects, features, benefits, and advantages of the present invention shall become apparent from the detailed drawings and descriptions provided herein.

### BRIEF DESCRIPTION OF THE DRAWINGS

FIG. 1A is a front view of an implantable sensor located adjacent to a hip prostheses in wireless communication with an external receiver according to one embodiment of the present invention.

FIG. 1B is an enlarged view of the implantable sensor of FIG. 1A.

FIG. 1C is a schematic illustration of the implantable sensor of FIG. 1A.

FIG. 1D is an enlarged cross-sectional side view of a portion of a prepared bone.

FIG. 1E is a cross-sectional side view of the implantable sensor of FIG. 1A implanted within the prepared bone of FIG. 1D and a portion of the hip prosthesis of FIG. 1A engaged with the bone of FIG. 1D.

FIG. 2A is an enlarged front view of an implantable sensor located adjacent to a hip prostheses according to one embodiment of the present invention.

FIG. 2B is an enlarged side view of the implantable sensor of FIG. 2A.

FIG. 2C is an enlarged cross-sectional side view of a portion of the hip prosthesis of FIG. 2A.

FIG. 2D is an enlarged cross-sectional side view of the implantable sensor engaging the engagement area of the hip prosthesis and an adjacent bone.

FIG. 3 is a schematic illustration of the implantable sensor and external receiver of FIG. 2A, where the implantable sensor is in wireless communication with the external receiver.

FIG. 4 is a flow chart illustrating use of the implantable sensor and external receiver of FIG. 2A.

FIG. 5A is a perspective view of an implantable pedometer located in a first position of an ACL reconstruction according to one embodiment of the present invention.

FIG. 5B is a perspective view of implantable pedometers located in second and third positions of an ACL reconstruction.

FIG. 6A is an enlarged view of an implantable sensor according to one embodiment of the present invention.

FIG. 6B is an enlarged cross-sectional side view of a portion of a hip prosthesis.

FIG. 6C is a cross-sectional side view of the implantable sensor of FIG. 6A engaged with the portion of the hip prosthesis of FIG. 6B and each engaged with a bone.

FIG. 7A is a cross-sectional view of an implantable sensor according to one embodiment of the present invention attached to a portion of an exterior surface of a hip prosthesis.

FIG. 7B is an enlarged cross-sectional view of the implantable sensor and exterior surface of FIG. 7A.

FIG. 8A is a front view of an implantable sensor located within a hip prostheses according to one embodiment of the present invention.

FIG. 8B is an enlarged cross-sectional view of the implantable sensor and hip prosthesis of FIG. 8A.

FIG. 8C is a cross-sectional view of a plurality of implantable sensors according to the present invention disposed within a hip prosthesis.

FIG. 9 is a cross-sectional view of a two-part implantable sensor system according to one embodiment of the present invention shown spaced apart from a portion of a hip prosthesis.

FIG. 10 is schematic illustration of an implantable sensor according to one embodiment of the present invention.

FIG. 11A is a cross-sectional view of an implantable sensor according to one embodiment of the present invention being implanted via a cannula.

FIG. 11B is the implantable sensor of FIG. 10A shown in an implanted position.

FIG. 12A is an enlarged cross-sectional side view of an implantable sensor according to one embodiment of the present invention.

FIG. 12B is a cross-sectional view of the implantable sensor of FIG. 12A engaged with a portion of an implanted hip prosthesis.

FIG. 13A is an enlarged cross-sectional side view of an implantable sensor according to one embodiment of the present invention.

FIG. 13B is a cross-sectional view of the implantable sensor of FIG. 13A engaged with a portion of an implanted hip prosthesis.

### DESCRIPTION OF THE PREFERRED EMBODIMENTS

For the purposes of promoting an understanding of the principles of the present invention, reference will now be made to the embodiments illustrated in the drawings, and specific language will be used to describe the same. It will nevertheless be understood that no limitation of the scope of the invention is intended. Any alterations and further modifications in the described devices, instruments, methods, and any further application of the principles of the invention as described herein are contemplated as would normally occur to one skilled in the art to which the invention relates.

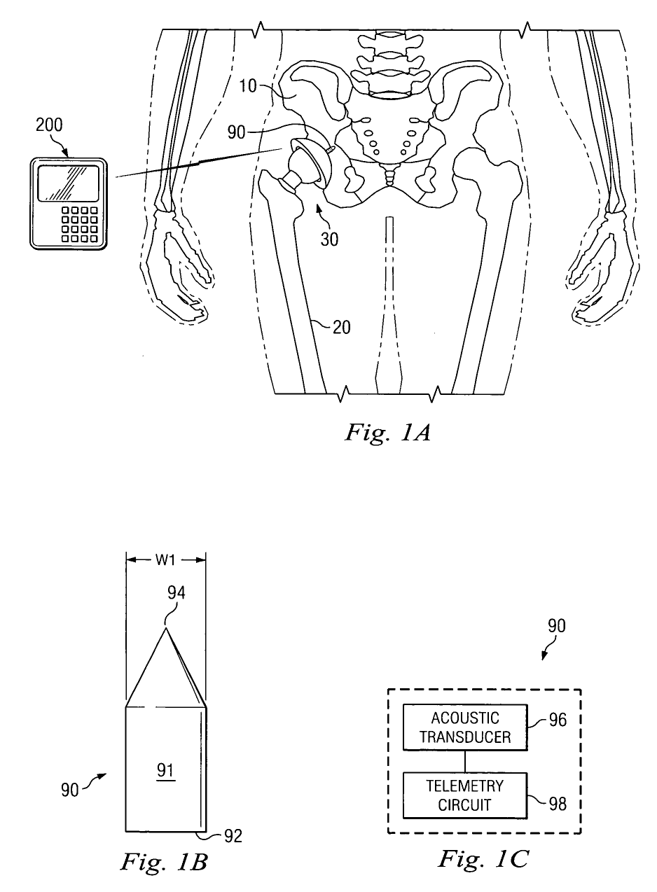

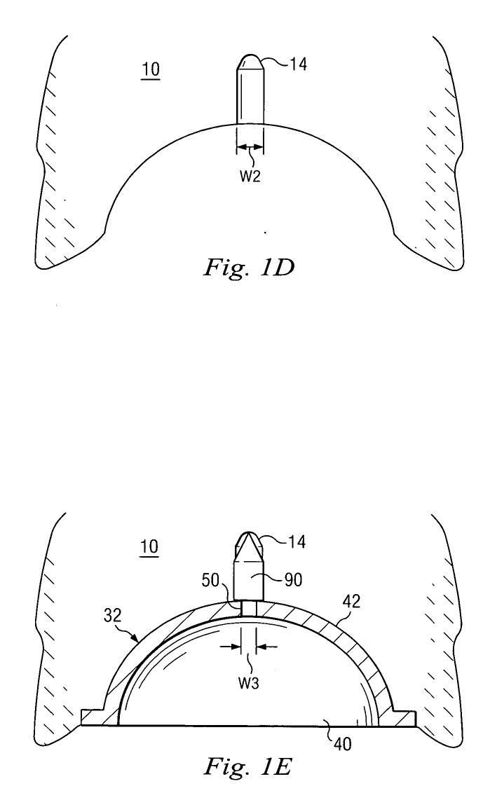

Referring now to FIGS. 1A-1E, shown therein is an implantable sensor 90 for monitoring changes in bone density in the bony areas 10, 20 around a hip implant or prosthesis 30 according to one aspect of the present invention. In particular, the sensor 90 is configured for detecting the onset of osteolysis and the development of osteolytic lesions. The hip prosthesis 30 being monitored includes an acetabular component 31 and a femoral component 33. The acetabular component 31 comprises an acetabular cup 32 configured for engagement with a prepared portion of the patient's acetabulum 10. As shown in FIG. 1E, acetabular cup 32 includes an opening 50 adapted to engage an insertion tool for driving the cup into position. The acetabular cup 32 also has a substantially spherical internal surface 40 and an exterior surface 42. The femoral component 33 comprises a head 34 and a stem 36. The femoral head 34 is configured for movable engagement with the internal surface 40 of the acetabular cup 32 so as to create ball-in-socket motion. The stem 36 of the femoral component is adapted for engaging a proximal portion 22 of the patient's femur 20. The ball-in-socket motion between the femoral head 34 and the acetabular cup 32 simulates the natural motion of the patient's hip joint.

FIG. 1A shows the implantable sensor 90 in wireless communication with an external device 200. The implantable sensor 90 is configured to detect and keep track of indicators associated with changes in tissue density. The implantable sensor 90 is also configured for wireless communication with the external device 200. Similarly, the external device 200 is configured for wireless communication with the implantable sensor 90. In particular, the external device 200 is adapted for retrieving and displaying, in human intelligible form, the tissue density data kept by the implantable sensor 90.

As discussed more fully below, it is fully contemplated that the sensor 90 may be disposed at a plurality of locations including, but not limited to, within a bone or tissue, attached to a bone or tissue, adjacent to a bone or tissue, within or integral to an artificial implant, attached to an artificial implant, adjacent to an artificial implant, or any combination of these locations. In the current embodiment the sensor 90 is disposed adjacent the hip implant 30 and partially within bone portion 10. Where the sensor 90 is adapted for being disposed at least partially within bone, it is contemplated that the sensor may be shaped or coated in a substance to facilitate bone growth and incorporation of the sensor into the bone. The sensor 90 is shown positioned adjacent the acetabular cup 32. However, the sensor 90 may also be disposed adjacent the femoral stem 36 of the hip implant 30. There are a plurality of other locations for the sensor 90 adjacent to the hip implant 30 that are adequate for monitoring changes in tissue density of the surrounding bone 10, 20. The precise locations available for placement of the sensor 90 will depend upon the type of sensor or transducer being utilized.

FIGS. 1B-1D shows in more detail the sensor 90 adapted for being disposed at least partially within a bone 10. The sensor 90 includes a main body 91 having a width W1, an implant engagement portion 92, and a bone engagement portion 94. In the illustrated embodiment, the bone engagement portion 94 is substantially similar to a bone nail. However, bone engagement portion 94 and the sensor 90 may be of any shape or form adapted for placement within a portion of a bone 10. In one embodiment, the sensor 90 is substantially shaped like a coin and adapted for placement within a portion of bone.

FIG. 1C shows a prepared opening 14 in the bone 10. The prepared opening 14 has a width W2 that is slightly smaller than width W1 of the sensor 90. The prepared opening 14 and its width W2 are configured such that the sensor 90 may be press-fit into the bone 10. It is contemplated that after the sensor 90 has been press-fit into the prepared opening 14 that it may then be sealed into the bone. The sensor 90 may be sealed into the bone using a variety of techniques. These sealing techniques may include, but are not limited to, fibrin glue, PMMA, collagen, hydroxyappetite, bi-phasic calcium, resorbable polymers or other materials suitable for implantation. Additionally or alternatively, the sensor 90 may be sealed into the bone by a later implanted implant, or any combination of these techniques. For example, the sensor 90 may be sealed in by any of the above mentioned materials in combination with an additional implant to provide enhanced fixation. In this manner, the sensor 90 may be implanted either prior to the implantation of an implant or as a stand alone unit—where no implant is to follow.

FIG. 1D shows the sensor 90 press-fit into the prepared opening 14 of the bone 10. Also shown is an implanted acetabular cup 32 having an inner surface 40, an external surface 42, and a driver opening 50. The external surface 42 of the acetabular cup 32 engages the bone 10. Driver opening 50 has a width W3 that is smaller than width W2 of the prepared opening 14 and, therefore, smaller than the width W1 of the sensor 90. In this manner the acetabular cup 32 may be used to seal the sensor 90 into the bone. If the sensor 90 was to come loose from the prepared opening 14 it would still not be dislodged as the acetabular cup would keep it in place. It is not necessary for driver opening 50 to seal the sensor 90 into place, other portions of the acetabular cup 32 may be used.

As shown in FIG. 1C, the sensor 90 includes an acoustic transducer 96 and a telemetry circuit 98. The acoustic transducer 96 is adapted for detecting indicators of tissue density. The telemetry circuit 98 is adapted for providing power to the acoustic transducer 96 and transferring the detected indicators to an external device 200. It is contemplated that the telemetry circuit will provide power to the acoustic transducer via inductive coupling or other known means of passive power supply. It is also contemplated that the external device 200 may be utilized to provide the power to the sensor 90 through coupling. That is, the sensor 90 may be externally powered. Further, this allows the sensor 90 to remain in a dormant state whenever an external power supply is not available and then become active when the external power supply is present. In this manner, the sensor 90 does not require a dedicated power supply such as a battery. This allows the sensor 90 to be much smaller than would otherwise be possible with a dedicated power supply, which in turn allows placement of the sensor in more locations without interfering with body mechanics or functions.

It is contemplated that the sensor may be utilized to detect indicators of tissue density over a regular interval such as every 6 months or every month as determined by the treating physician. In this regard, it is contemplated that the patient may return to the doctor's office for each reading. At such time the doctor would place the external device 200 in the vicinity of the sensor 90. Through inductive coupling via the telemetry unit 98 the sensor 90 would be powered by the external device 200. The acoustic transducer 96 would then take a reading by detecting indicators of tissue density. This reading would then be relayed to the external device 200 via the telemetry circuit 98. The reading may then be analyzed and appropriate medical treatment may be taken. It is also contemplated that the patient may obtain these readings without a need to go to the doctor's office. For example, the patient may be provided with the external device 200 that is capable of providing power to the sensor 90, obtaining the readings, and then relaying the readings on to the doctor's office. For example, the external device may transfer the readings to the doctors office via a phone line or computer network. It is contemplated that a system similar to that of Medtronic's CareLink may be utilized.

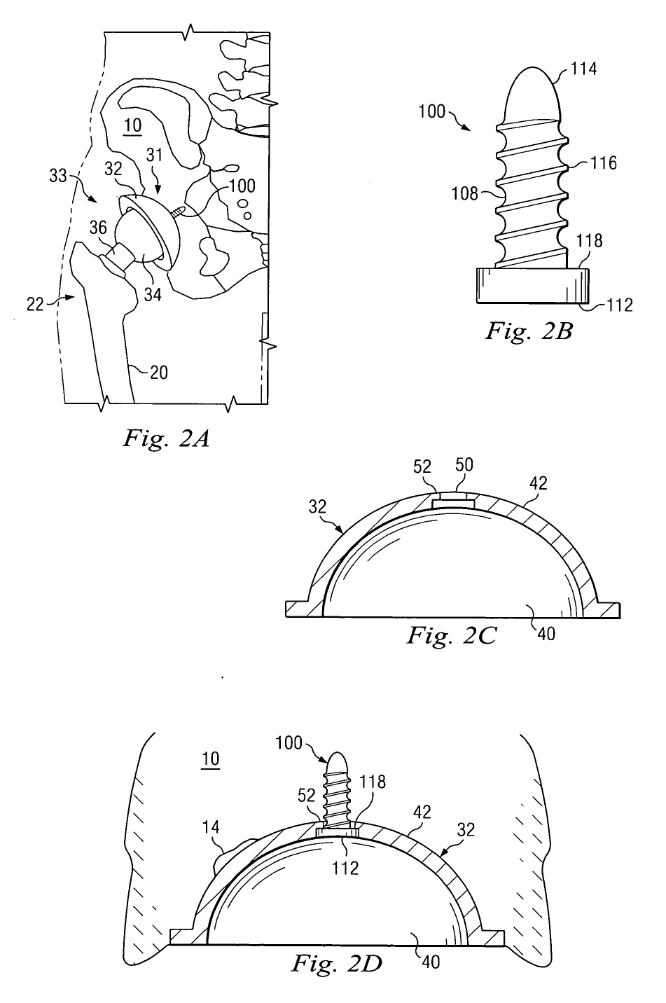

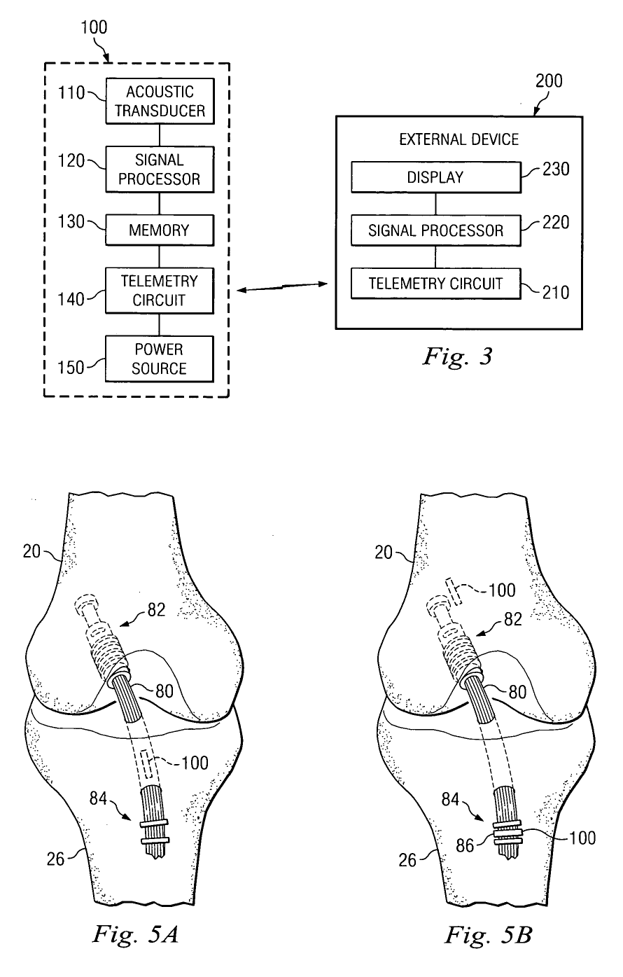

Now referring to FIGS. 2A-2D, a sensor 100 is disposed external to the acetabular cup 32. Sensor 100 may be substantially similar to sensor 90. In the illustrated embodiment, sensor 100 has a first portion-adjacent to the acetabular cup—and a second portion—extending into the bone 10 adjacent to the acetabular cup 32. FIG. 2B shows the sensor 100 in more detail. The sensor 100 includes a main body 108. A head 112 of the sensor 100 includes a flange portion 118. A leading end 114 of the sensor 100 is adapted for being disposed within bone. To facilitate bone engagement the sensor 100 includes threads 116. The threads 116 are configured such that the sensor 100 may act as a bone screw. Thus, threads 116 should be of an appropriate size and shape to encourage bone engagement.

As shown in FIG. 2C, opening 50 of the acetabular cup 32 includes an internal flange 52 of reduced diameter. The flange portion 118 of the sensor 100 is adapted for engaging the internal flange 52 of opening 50. The inner surface 40 of the acetabular cup 32 is adapted for movable engagement with the femoral head 34 of the hip implant 30. Flange portion 52 is recessed with respect to inner surface 40 of the acetabular cup 32 so that when flange portion 118 is engaged with flange 52 the head 112 substantially aligns with internal surface 40 and does not inhibit the movable engagement between the femoral head 34 and the inner surface 40. FIG. 2D shows sensor 100 attached to the acetabular cup 32 and engaged with the bone 10 for detection of changes in tissue density within the bone 10, such as the development of osteolytic lesion 14.

In the illustrated embodiment, it is also contemplated that the sensor 100 may be implanted in a surgical procedure after the acetabular cup 32 has been implanted. It is also contemplated that the sensor 100 may be implanted when the acetabular cup 32 is implanted. It is also contemplated that the sensor 100 may be implanted into a bone without engaging a portion of a previously implanted implant. That is, the sensor 100 may be a stand-alone unit.

The implantable sensor 100 includes an acoustic transducer 110, a signal processor 120, a memory unit 130, a telemetry circuit 140, and a power supply 150. While the implantable sensor 100 is described as having a separate signal processor 120, it is fully contemplated that the function of the signal processor, described below, may be incorporated into either the transducer 110 or the memory 130, eliminating the need for a separate signal processor. Similarly, it is fully contemplated that the functions of the various components of the sensor 100 may be combined into a single component or distributed among a plurality of components. Further, it is fully contemplated that the sensor 100 may include other electronics and components adapted for monitoring indicators of tissue density and changes in tissue density.

The implantable sensor 100 may function in a variety of ways. Under one approach the sensor 100 may use a type of comparative analysis to determine changes in tissue density. That is, an initial baseline or threshold range of signals will either be determined by the sensor itself or provided to the sensor by the caretaker. Then the sensor 100 will monitor the indicators of tissue density and when the signals detected are out the threshold range the sensor will store those signals in its memory 130. Then this data may be extracted by the caretaker via external device 200. With this data the caretaker may then choose the appropriate treatment plan. For example, the caretaker may choose to have the patient undergo additional examinations such as a CT scan or an x-ray. Either based on the additional examinations or other factors, the caretaker may instead or in addition choose to adjust the threshold range.

It is fully contemplated that a treating physician may want to change what the sensor considers the normal range of signals overtime. For example, as an artificial implant is incorporated into the body the signals associated with tissue density near the bone-implant connection point will change until the implant is fully integrated. Once the implant is fully integrated, the normal range of signals may be consistent for a period of months or years, but still may change over time requiring modification of the range. Thus, it is contemplated that the sensor 100 be programmable, self-learning, or both.

Self-learning implies that the sensor 100 is able to determine the proper range of signals by monitoring the signals over a period of time and then via algorithms in its signal processing unit decide on the range of signals indicative of normal tissue density. In this regard, it is fully contemplated that the caretaker may be able to override the determinations made by the sensor 100 by programming in the thresholds or, on the other hand, the caretaker may reset the sensor's determinations and simply have the sensor recalculate the proper range based on current signals detected. Thus, as described above when an implant becomes fully integrated the caretaker may decided to reset the self-learning sensor so that the ranges are based on the signals associated with the fully integrated implant.

In regards to setting the ranges, it is contemplated that the patient may be instructed through a series of movements such as sitting down, standing up, walking, climbing stairs, or cycling with the sensor 100 detecting the associated indicators of tissue density. Based on the sensed signals, the sensor threshold ranges may be set for operation. The acoustic signals produced by these and other movements may be detected within a bone being monitored as cortical bone is known to be acoustically conductive. Thus, instructing the patient through many of the normal motions and movements of everyday life may provide a good variety of signals that may be used to base the normal signal range upon. Over time, the patient may again be put through a similar series of movements to reset or recalibrate the sensor 100 as seen fit by the caretaker.

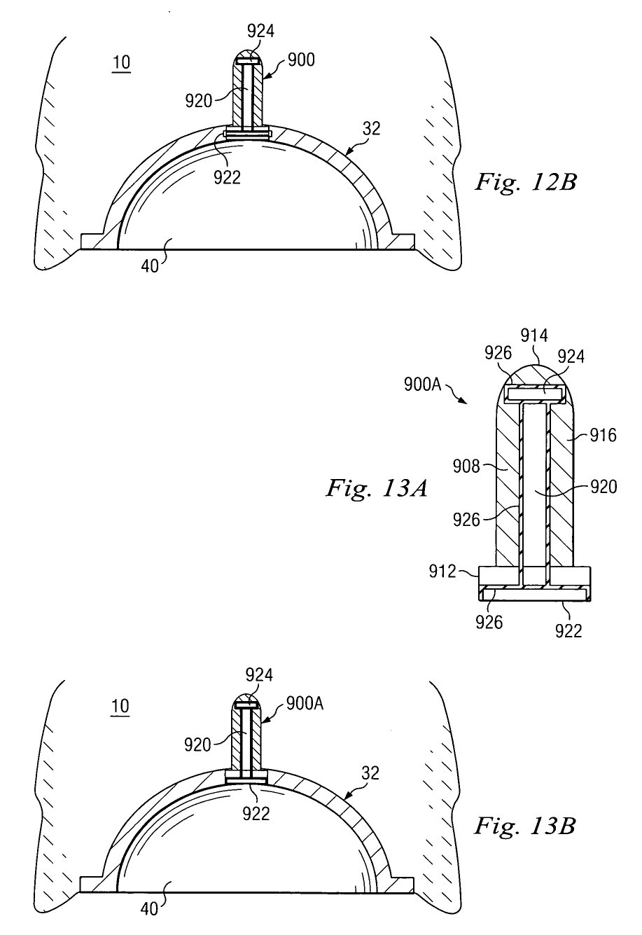

Under another approach, the sensor 100 may function by monitoring for signals determined to be associated with the onset of osteolysis or other changes in tissue density. For example, there are certain acoustic sounds and vibrations associated with osteolytic lesions. The sensor 100 may be configured to detect and recognize these acoustic signals. For example, the sensor 100 may utilize various filters, amplifiers, and algorithms to remove background noise and focus on the detection of the signals indicative of osteolysis or other changes in tissue density. Though in the currently described embodiment the sensor 100 is an acoustic sensor, it is also contemplated, and described more fully below with respect to FIGS. 12A-12C, that the sensor 100 may utilize impedance to detect changes in tissue density.

In the case of an acoustic sensor as in the present embodiment, the acoustic transducer 110 is configured for detecting sounds and acoustic waves indicative of tissue density. Under one approach if the detected signal exceeds the normal range of signals as determined by the signal processor 120, then the signal will be stored in the memory 130. In this regard, the signal processor 120 may be configured to determine the parameters or threshold levels of signal ranges for detection by the sensor 100. The signal processor 120 may set parameters such as the amplitude, frequency range, or decibel level required before a signal is considered an indication of a change in tissue density. The range and parameter settings may be configured so as to increase the accurate detection of changes in tissue density.

The memory 130 is configured to store data it receives from the signal processor 120 that is either outside the normal signal range or within the range of signals being detected. It is fully contemplated that the memory 130 may utilize known compression algorithms and functions to save on memory and size requirements. In this regard, it is also contemplated that the memory 130 may store additional data with respect to each signal such as a timestamp, the specific characteristics of the signal, or any other relevant data. In this respect, the signal processor 120 and memory 130 may be configured to keep the various types of data the orthopedic surgeon or treating physician would like to have to monitor tissue density.

The implantable sensor 100 also includes a telemetry circuit 140. The telemetry circuit 140 is connected to the memory 130 and is adapted for sending the data stored in the memory outside of the patient's body to an external device 200. In particular, the telemetry circuit 140 is adapted for communicating wirelessly with the telemetry circuit 210 of the external device 200. There are several types of wireless telemetry circuits that may be employed for communication between the implantable sensor 100 and the external device 200. For example, RFID, inductive telemetry, acoustic energy, near infrared energy, “Bluetooth,” and computer networks are all possible means of wireless communication. In the present embodiment, the telemetry circuits 140, 210 are adapted for RFID communication such that the telemetry circuit 140 is a passive RFID tag. Using a passive RFID tag helps limit the power requirements of the telemetry circuit 140 and, therefore, the implantable sensor 100 yet still allows wireless communication to the external device 200.

Supplying the power requirements of the implantable sensor 100 is a power source 150. In the current embodiment, the power source 150 is a battery. In this manner the sensor may be internally powered. The battery power source 150 may be a lithium iodine battery similar to those used for other medical implant devices such as pacemakers. However, the battery power source 150 may be any type of battery suitable for implantation. The power source 150 is connected to one or more of the transducer 110, the signal processor 120, the memory 130, or the telemetry unit 140. The battery 150 is connected to these components so as to allow continuous monitoring of indicators of tissue density. It is fully contemplated that the battery 150 may be rechargeable. It is also contemplated that the battery 150 may be recharged by an external device so as to avoid the necessity of a surgical procedure to recharge the battery. For example, in one embodiment the battery 150 is rechargeable via inductive coupling.

In the current embodiment, the sensor 100 is passive. However, it is fully contemplated that the sensor 100 be active. Where the sensor 100 is active, the transducer 110 may use a pulse-echo approach to detecting bone density. For example, utilization of ultrasonic waves in a pulse-echo manner to determine tissue density is fully contemplated. In that case, the transducer 110 would utilize power from the power source 150 to generate the pulse signal. In the current embodiment, however, the transducer 110 may use the power source 150 to facilitate the sending of signals to the signal processor 120. The signal processor 120, in turn, may use the power source 150 to accomplish its filtering and processing and then send a signal to the memory 130. The memory 130 will then use the power source 150 to store the signal and tissue density data.

In other embodiments the power source 150 may also be connected to the telemetry circuit 140 to provide power to facilitate communication with the external device 200. However, in the present embodiment the telemetry circuit 140 does not require power from the power source 150 because it communicates with the external receiver 200 utilizing a passive RFID tag or other inductive coupling means of communication. Further, the power source 150 may be connected to other electronic components not found in the current embodiment. It is fully contemplated that the power source 150 may include a plurality of batteries or other types of power sources. Finally, it is also contemplated that the implantable sensor 100 may be self-powered, not requiring a separate power supply. For example, a piezoelectric transducer may be utilized as the acoustic transducer 110 such that signals detected by the transducer also provide power to the sensor 100. The piezoelectric transducer could detect the signal and converts it into an electrical signal that is passively filtered and stored only if it satisfies the signal thresholds. Then, as in the current embodiment, the sensor 100 may utilize a passive RFID tag or other passive telemetry unit to communicate the tissue density data with an external device. Thus, allowing the sensor 100 to function without a dedicated or continuously draining power source. Similarly, the sensor 100 may utilize a piezoelectric or electromagnetic power source that is not used as the acoustic transducer 110. For example, such power sources could utilize patient motion to maintain a power supply.

The external device 200 receives the tissue density data from the implantable sensor 100 via communication between the telemetry circuit 140 of the sensor and the telemetry unit 210 of the external device. Then a signal processor 220 converts or demodulates the data. The converted data is output to a display 230 where it is displayed in human intelligible form. The conversion and processing of the data may be tailored to the specific liking of the surgeon. For example, the display of data may simply be a number representing the number of signals recorded by the memory 130 indicating the number of signals outside the normal range that were detected. Similarly, the display of data may be a bar graph having a height or length representing the number of signals detected. Further, the display may show a detailed chart of specific information for each signal detected outside of the threshold range. These various display examples are for illustration purposes only and in no way limit the plurality of ways in which the tissue density data may be displayed in accordance with the present invention.

Utilizing the sensor 100 to detect indicators of changes in tissue density may have numerous applications. The detected changes may be used to predict the onset of osteolysis and osteolytic lesions. Under such an approach, early detection will allow the treating physician to treat the affected regions before the problem escalates. In particular, early detection may prevent the need for a later revision surgery if the detected problem is treated promptly. Under another approach described more fully below, the sensor 100 may be utilized to monitor and track the healing process and coordinate post-operative treatment and physical therapy accordingly.

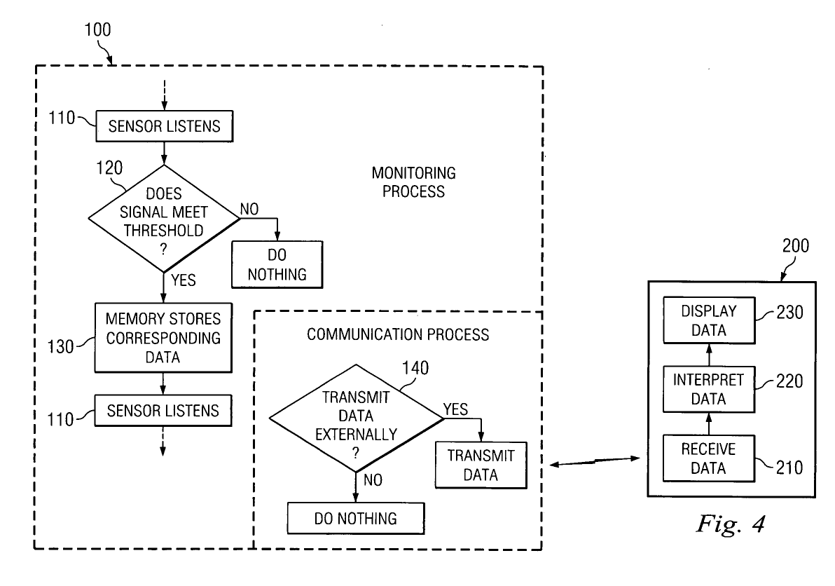

FIG. 4 illustrates a possible flow chart for tissue density data detection, processing, and output employing the current embodiment of the invention. The internal monitoring process occurring within the sensor 100 constitutes a continuous loop of monitoring and storing the tissue density data. The acoustic transducer 110 listens for signals indicative of tissue density. Upon detecting a signal the signal processor 120 determines if the signal meets the preset parameters. If the signal does not meet the thresholds, then the signal processor 120 does nothing. If the signal does meet the parameters, then the signal processor 120 passes along a signal to the memory 130. The memory 130 stores the tissue density data accordingly. The internal process continues as the acoustic transducer 110 listens for the next tissue density signal.

Also within the sensor 100, a communication process is underway. The telemetry unit 140 awaits communication from the external device 200 requesting transmission of the tissue density data. If the telemetry unit 140 receives such a request, then the telemetry unit 140 transmits the tissue density data to the telemetry unit 210 of the external receiver 200. From there the signal processor 220 converts or demodulates the transferred data and the display 230 displays the demodulated data in a human intelligible form. At this point the surgeon or caretaker can review the tissue density data and take the appropriate medical action as they see fit.

Though not illustrated, it is also contemplated that the external device 200 may reset the tissue density data stored within the sensor 100. For example, the external receiver 200 may be configured to reset or clear the memory 130 upon extraction of the tissue density data. The external device 200 may clear the memory 130 of the sensor 100 by utilizing communication between the telemetry circuits 140, 210. However, it is not necessary for the external device 200 to clear the data of the sensor 100. For example, a treating physician may wish to keep a running count of signals detected outside the normal range in the memory 130 rather than resetting the sensor 100 after each data extraction.

Described below are numerous components of the external receiver in accordance with the present invention. These components illustrate the various types of electronic and non-electronic components that may be utilized by the external receiver. These descriptions are exemplary of the type of components that may be employed by the external receiver, but in no way are these illustrations intended to limit the types or combinations of electronic and non-electronic components that may be utilized in accordance with the present invention.

The external receiver may include components such as a telemetry unit, a signal processor, a calibration unit, memory, an indicator, and a networking interface. The telemetry unit is adapted for communication with the implantable sensor in accordance with the present invention. Thus, the telemetry unit is configured to extract tissue density data from the sensor. The telemetry unit may obtain data from the sensor through a variety of wireless communication methods such as inductive coupling, capacitive coupling, radio frequency, personal computer networking, Bluetooth, or other wireless means. Though the preferred method of communication is wireless, it is also contemplated that the external receiver may be in selective wired communication with the implantable sensor.

Once the data is obtained by the external receiver using the telemetry unit, the data is processed by the signal processor. The degree and type of data processing is dependant on both the data obtained from the implantable sensor and the desires of the treating doctor. The data processing performed by the signal processor may range from simple conversion of tissue density data into a human sensible form to complex analysis of the usage data via spectral analysis. Further, the data processing performed by the signal processor may only be a first step of processing. The processed data of the external receiver may be output to a more powerful or specialized signal processing unit where additional processing takes place. This additional signal processing unit may be located either within the external receiver itself or in a separate external device such as a personal computer.

The signal processor is adapted for converting the data into a form that may be utilized by an indicator. The indicator may be any type of device or interface that can output the data in human intelligible form. For example, the indicator may be a visual display, speaker, or any other indicator or output means. It is contemplated that the indicator may be composed of a plurality of output mechanisms instead of a single device.

The external receiver may also include a calibration circuit. The calibration circuit is adapted for configuring a configurable signal processor of an implantable sensor. The external receiver may set, restore, or change such aspects of the configurable signal processor as the predetermined criteria for keeping sound recordings, the type of tissue density data to be kept, the preset thresholds for signals indicative of normal tissue density, or any other setting related to the performance of the configurable signal processor. It is fully contemplated the calibration circuit may utilize the telemetry circuits of the sensor and external receiver to communicate with the configurable signal processing unit. However, it is also fully contemplated that the calibration circuit and the configurable signal processing unit may have a separate dedicated means of communication.

The external receiver may also include a memory unit. The memory unit may be adapted for multiple uses. First, the memory unit may be adapted for permanent storage of tissue density data obtained from the implantable sensor. Thus, the memory unit may store data obtained at various times from the implantable sensor so the data may later be reviewed, compared, or analyzed. Second, the memory unit may be adapted for temporary storage of tissue density data obtained from the implantable sensor. In this case, the memory unit will store the data until it is either discarded or transferred for permanent storage. For example, the data may be transferred from the memory unit of the external receiver via a networking interface to a network or computer for permanent storage.

When present, the networking interface provides a means for the external receiver to communicate with other external devices. The type of network utilized may include such communication means as telephone networks, computer networks, or any other means of communicating data electronically. The networking interface of the external receiver could obviate the need for the patient to even go into the doctor's office for obtaining implant usage data. For example, the patient could utilize the external receiver to obtain the usage data from the implantable sensor on a scheduled basis (e.g. daily, weekly, monthly, etc.). Then, utilizing the networking interface the patient could send this data to the treating doctor. The networking interface may be configured to directly access a communication network such as a telephone or computer network for transferring the data. It is fully contemplated that the computer network be accessible by a treating physician for reviewing implant usage data of the patient without requiring the patient to make an actual visit to the doctor's office. The networking interface may be similar to the CareLink system from Medtronic, Inc.

Further, it is also contemplated that any communication between the external receiver and the computer network may be encrypted or otherwise secured so as protect the patient's privacy. It is also contemplated that the networking interface may be configured for communication with a separate device that is adapted for accessing the communication network. For example, the networking interface may be a USB connection. The external receiver may be connected to a personal computer via the USB connection and then the personal computer may be utilized to connect to the communication network, such as the internet, for transferring the data to a designated place where the treating doctor may receive it.

Referring now to FIGS. 5A-5B, the sensor 100 may have particular uses as related to monitoring indicators of tissue density in and around the knee. For example, with ACL reconstruction surgery the sensor 100 may be utilized to monitor and track the healing process and coordinate post-operative treatment and physical therapy accordingly. The sensor 100 can detect indicators of tissue density related to the incorporation of the graft 80 into the femur and tibia. As shown in FIG. 5A, it is contemplated that the sensor 100 may be disposed within the graft 80. As shown in FIG. 5B, it is also contemplated that the sensor 100 may be disposed adjacent the grafting area—as shown in the upper or femur grafting portion 82—or the sensor may be incorporated into the fixation device 86 such as a bone screw or other means of securing the graft—as shown in the lower or tibia grafting portion 84. The sensor 100 may be utilized to determine the relative degree of incorporation of the graft and help determine what treatment is available for the patient. For example, the sensor 100 may be utilized to determine when the graft is sufficiently incorporated into the femur and tibia to allow full weight bearing on the knee.

The sensor 100 may provide tissue density data to the doctor or physical therapist allowing the treatment and physical therapy to the be tailored to the specific recovery speed of the patient. In this regard, it is also contemplated that the sensor 100 may be used to determine the rate of healing for each patient. That is, the sensor 100 may be used to predict the state of healing at a later time. For example, based on the status of healing at a first time compared to the status of a standard healing process the treating physician may project the state of healing for the particular patient at a later time. This may be particularly useful in the case of a patient who needs to speed up the recovery time as much as possible without reinjuring the knee, such as a professional athlete. Similarly, the sensor 100 may also provide early evidence of incorporation problems and allow the surgeon to remedy these problems earlier.

It is also contemplated that the sensor 100 may also be used for monitoring other aspects of the knee not associated with ACL reconstruction surgery. For example and without limitation, the sensor 100 may be used to monitor tissue density changes of the meniscus, osteochondral cartilage, or articular cartilage. The sensor 100 may also be used to sense the amount of synovial fluid, density of synovial fluid, and the pressure of synovial fluid in the synovial capsule; these determinations may be particularly advantageous in partial joint replacements. Also it is fully contemplated that the sensor 100 may be utilized for similar tissue density monitoring in parts of the body other than the knee. Further, the sensor 100 may be utilized to monitor the density of tissue adherent to bone. For example, the sensor 100 may be used to monitor the connections between ligaments and bone or tendons and bone. The sensor 100 may also be utilized to determine the density of muscle tissue surrounding the bone.

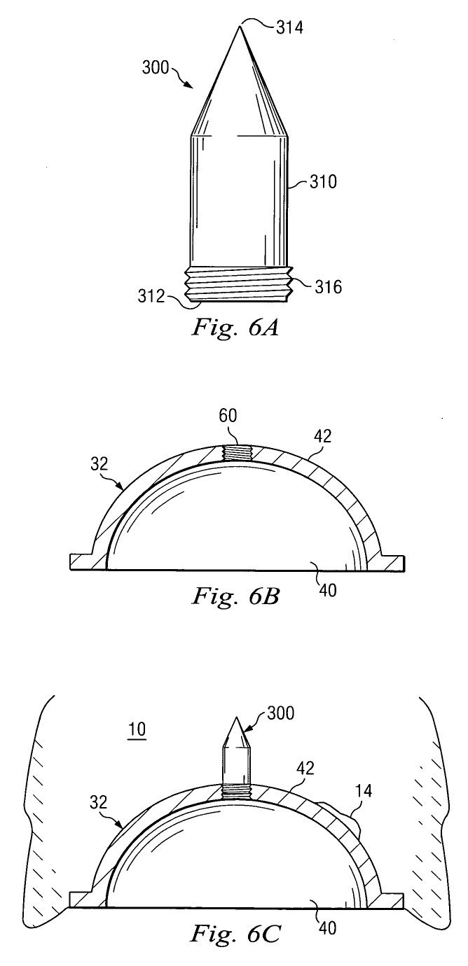

FIGS. 6A-6C show a sensor 300 adapted for being disposed at least partially within a bone 10. The sensor 300 includes a main body 310, an implant engagement portion 312, and a bone engagement portion 314. In the illustrated embodiment, the bone engagement portion 314 is substantially similar to a bone nail. The implant engagement portion 312 includes machine threads 316. The machine threads 316 are adapted for engaging a threaded portion of an implant. For example, as shown machine threads 316 may be adapted for engaging a threaded driver portion 60 of an acetabular cup 32. The inner surface 40 of the acetabular cup 32 is adapted for movable engagement with the femoral head 34 of the hip implant 30. The implant engagement portion 312 and the threaded driver portion 60 are configured such that when the two portions are threaded together the movable engagement between the femoral head 34 and the inner surface 40 is not inhibited. In this regard, it is contemplated that the implant engagement portion 312 be shaped to substantially match the contours for the inner surface 40 once attached to the acetabular cup 32. FIG. 6C shows the sensor 300 attached to the acetabular cup 32 and engaged with the bone 10 for detection of changes in tissue density within bone 10, such as the development of osteolytic lesion 14.

In the illustrated embodiment, it is contemplated that the sensor 300 may be implanted after the acetabular cup 32 has been implanted. Under one approach, the sensor 300 may be impacted or otherwise advanced into the bone 10 until the threads 316 of the implant engagement portion 312 are in a position to be threaded into the threaded driver portion 60. Then the sensor 300 may be rotated until the threads 126 and threaded driver portion 60 are fully threaded together. It is contemplated that the implant engagement portion 312 may include a cross-shaped driver opening or other mechanism to facilitate rotation of the sensor 300 by another device such as a driver. Under another approach, the sensor 300 may be driven into a bone without engaging an implant.

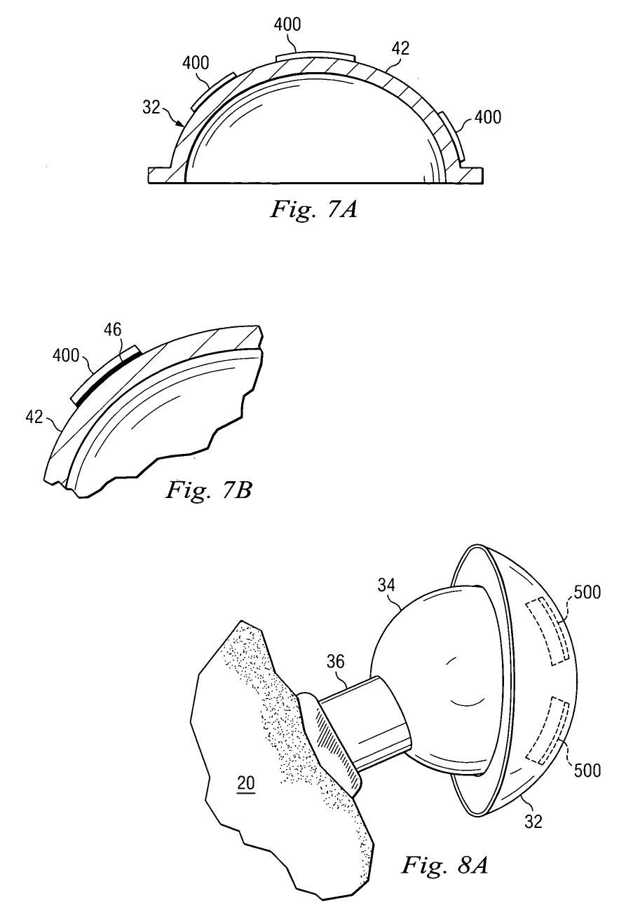

Referring now to FIGS. 7A-7B, shown therein is an alternative embodiment of a sensor 400 for monitoring tissue density in accordance with another aspect of the present invention. FIGS. 7A and 7B show an implantable sensor 400 attached to a surface 42 of an acetabular cup 32. It is contemplated that the sensor 400 may be associated with surface 42 without being fixedly mounted. However, it is also contemplated that, as shown, the sensor 400 may be attached to the surface 42 of the acetabular cup 32 by any reliable means. One means of attachment is fibrin glue. Fibrin glue may be utilized to secure the sensor 400 to the surface 42. As shown in FIG. 7B, a very thin interface layer 46 of fibrin glue may be used to glue the sensor 400 to the implant. Interface layer 46 is shown much thicker for illustration purposes only. It is contemplated that the sensor may be attached to a portion of the implant prior to implanting the implant. However, it is also contemplated that the sensor be attached to a portion of the implant at some time after implantation of the implant.

FIG. 7A shows a plurality of sensors 400 being utilized. It is fully contemplated that a plurality of sensors 400 may be utilized to monitor changes in tissue density. In this regard, the plurality of sensors 400 may work together to form a type of sensing network. Under such an approach the plurality of sensors 400 may be configured to recognize not only changes in tissue density, but also where those changes are occurring. Utilizing a plurality of sensors allows a spatial relationship to be determined based on the location of the sensors and then based on the signals detected the location of any tissue density changes can be mapped accordingly. The plurality of sensors 400 essentially may triangulate the location of the signals. In this regard the plurality of sensors 400 may be spaced apart by at least 5 mm to allow for accurate triangulation. In one embodiment, the plurality of sensors 400 may be spaced apart by at least 20 mm. Thus, it is contemplated that the sensors may be placed in numerous arrangements. For example and without limitation, for monitoring the bone surrounding the hip joint the sensors may be placed on both sides of the iliac crest, within the femur and the acetabulum, within the acetabular cup and femoral stem, or separated within the acetabular cup.

In addition or alternatively, the plurality of sensors 400 may function as redundancies to one another. That is, rather than working together each individual sensor 400 would function independently. Then the data obtained by each sensor could be compared to the data obtained by the other sensors to make a determination of changes in tissue density. Under such an approach, the failing of a single sensor would not create a need to replace the sensor and therefore eliminate the need for an additional medical procedure. Further, it is fully contemplated that all of the sensors of the present invention may be utilized independently or as part of a plurality of sensors.

The plurality of sensors 400 and all other sensors of the present invention may be accelerometers. Further, accelerometers and other sensing means may be used in combination to form the plurality of sensors 400. An accelerometer can be utilized to detect vibrations. In the relation to the acoustic sensors previously described, it is contemplated that the vibrations detected by an accelerometer may be a result of the acoustic emissions or the producing cause of the acoustic emissions. Thus, in this respect it can be advantageous to use both an acoustic sensor and an accelerometer. Further, the accelerometer may be a single or multi-axis device. Also, a plurality of single-axis accelerometers—in the same or different axis—may be utilized to simulate the advantages found with a multi-axis accelerometer. For example, the use of a multi-axis accelerometer or a plurality of single-axis accelerometers may be used to produce vectored data to better differentiate between locations and types of bone lysis.

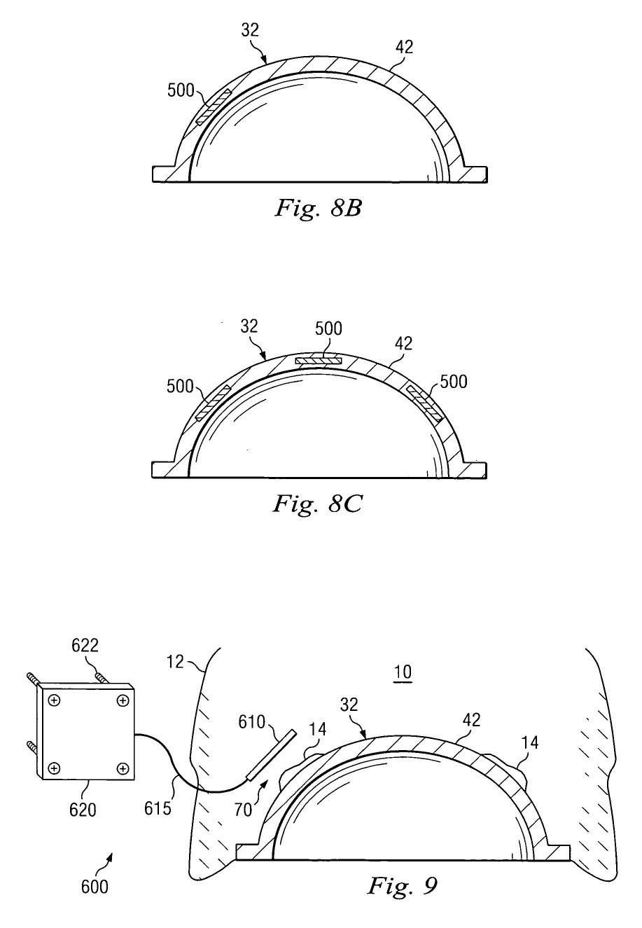

Referring now to FIGS. 8A-8C, shown therein is an alternative embodiment of a sensor 500 in accordance with the present invention. FIGS. 8A-8C show the sensor 500 disposed within the acetabular cup 32 of the hip implant 30. It is fully contemplated that the sensor 500 may also be disposed within the femoral head 34 or stem 36 of the hip implant 30. Further, it is contemplated that the sensor 500 may be disposed within a portion of the hip implant 30 during manufacture of the hip implant. However, where the sensor 500 is to be disposed within a portion of the hip implant 30, it is preferred that the sensor be adapted for placement within one of the portions of the hip implant 32, 34, 36 after manufacture of the hip implant. For example, the sensor 500 may be placed into an available opening of the implant or manually placed into a recess in the surface of the implant and then sealed into the implant. In this manner the sensor 500 may be utilized with the hip implant 30 regardless of the manufacturer of the hip implant. FIG. 8C illustrates that a plurality of sensors 500 may be disposed within the implant in accordance with the present invention.

Referring now to FIG. 9, shown therein is an alternative embodiment of a sensor for monitoring use of an implant in accordance with another aspect of the present invention. A sensor system 600 is shown in a position for monitoring the tissue density around the hip joint, and in particular for monitoring tissue density near an artificial acetabular cup 32. The sensor system 600 may be substantially similar to the other sensors described in accordance with the present invention. However, sensor system 600 includes a transducer 610 for insertion into a bone or tissue and a separate main housing 620. It is contemplated that the main housing 620 will contain the remaining components of the sensor system 600 such as a signal processor, memory unit, telemetry unit, power supply, and any other component. As illustrated, the main housing 620 is adapted to be positioned away from the transducer 610. Main housing 620 is located outside of the exterior bone surface 12 of bone 10. Main housing 620 may be attached to the bone 10 via anchoring elements 622, that may be such things as spikes or screws. Main housing 620 may also be adapted for positioning within soft tissue. Positioning the main housing 620 away from the transducer 610 allows the transducer, which may be miniaturized, to be placed in a desired location without requiring the additional space to house the remaining components of the sensor system 600. It is fully contemplated that the main housing 620 may be shaped similar to a cylinder or otherwise so as to facilitate implantation via a catheter.

Transducer 610 may be substantially cylindrical such that it can be delivered to the implantation site via a needle or catheter. In this respect, the transducer 610 may communicate with the components in the main housing 620 via a dedicated wire or lead 715, as shown. On the other hand, the transducer 610 may communicate with the components in the main housing 620 wirelessly. For example, the transducer 610 may utilize an RF transponder or other means of wireless communication to transfer information to the main housing 620.

Though the main housing 620 is shown as being disposed inside the body and near the hip joint, it is fully contemplated that the main housing may be disposed anywhere within communication range of the transducer 610. Thus, the main housing 620 is preferably located where it will not interfere with use of the joint nor interfere with any other body functions. Where the transducer 610 communicates with the components of the main housing 620 via the wire lead 615, the location of the main housing is limited by potential interference of both the wire and the main housing. Where the transducer 610 communicates with the components in the main housing 620 wirelessly, the position of the main housing 620 will be a function of the limits on the distance for wireless communication as well as any potential body function interference the main housing may cause. With sufficient wireless communication it is possible to position the main housing 620 externally. That is, the main housing 620 may be positioned outside the patient's body. Preferably, when disposed outside of the body the main housing 620 will be positioned in a location anatomically close to the transducer 610. Placing the main housing 620 as close to the location of the transducer 610 as possible helps to facilitate wireless communication. It is not necessary to place the main housing 620 near the transducer 610 if communication can be achieved from greater distances.

FIG. 9 shows the transducer 610 implanted within bone 10 near an acetabular cup 32 but spaced apart from the acetabular cup as illustrated by gap 70. Gap 70 is shown relatively large for the purposes of illustration. However, gap 70 may be much smaller than the thickness of the sensor or the implant. In the illustrated embodiment, it is contemplated that the sensor system 600 may be implanted percutaneously either prior to or after implantation of the acetabular cup 32. The size and shape of the components of the sensor system 600 may be adapted for insertion through a catheter, needle, or any other means of insertion. For example, it is contemplated that the transducer 610 be miniaturized to facilitate ease of placement in any desired location. Then utilizing a lead or wireless communication the transducer 610 may communicate with the main housing 620, which may be placed in less intrusive position for ease of implantation. Implanting the sensor system 600 may be a minimally invasive procedure. In this manner, the sensor system 600 may be utilized to monitor tissue density even prior to artificial joint replacement surgery without causing severe trauma to the patient or furthering injuring the tissue being monitored. Similarly, the sensor system 600 may be implanted after joint replacement surgery without requiring open surgery or otherwise compromising the healing process or integration of the implant into the body.

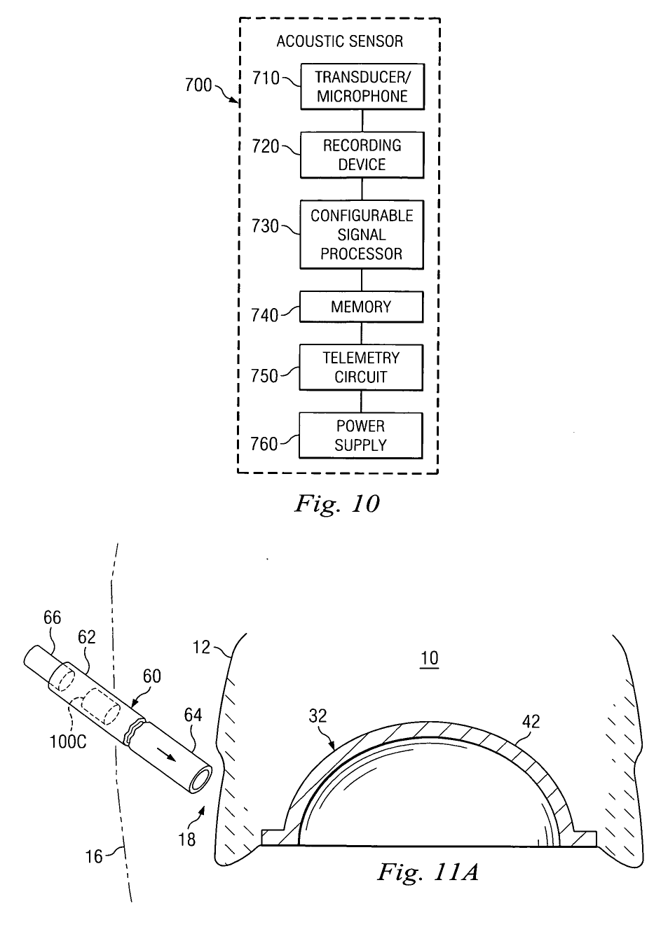

FIG. 10 shows an acoustic sensor 700 having a transducer 710, a recording device 720, a configurable signal processor 730, memory unit 740, a telemetry unit 750, and a power supply 760. Sensor 700 may be substantially similar to other embodiments of the present invention. As illustrated sensor 700 includes a recording device 720 and a configurable signal processor 730. In regard to the recording device 720, it is known that there are certain sounds indicative of patient activity. Specifically the pounding of walking and running may be sensed and recorded as an indicator of joint usage. Additionally, but not required, other sounds indicative of implant degradation may be detected. For example, associated with the wear of a hip implant are sounds of “play” or movement within the components of the hip implant itself or between the hip implant and the surrounding bone. This play may be characterized by a clicking sound caused by the worn hip implant socket. These various sounds may be used to monitor joint usage include natural and artificial joints as more fully described in a patent application entitled “Implantable Pedometer.” The United States patent application entitled “Implantable Pedometer,” attorney docket No. P22387/31132.428 filed on even date is incorporated herein by reference in its entirety.

Similarly, with the onset of osteolytic lesions the bone begins to create “mushy” or “soft” sounds with each step taken or other movement. As indicated above, osteolytic lesions are often caused by polyethylene wear debris from deteriorating implants. In this manner, the sensor 700 may be utilized for the detection of osteolytic lesions as well as for monitoring implant use. Thus, it is advantageous for the sensor 700 to include a means of detecting and recording these sounds for later review by a surgeon or other caretaker.

It is contemplated that the transducer 710 may be a microphone or other type of transducer that facilitates detection and recording of sounds indicative of tissue density. The transducer 710 is connected to the recording device 720 such that the recording device is able to store the sounds picked up by the transducer. However, due to a desire to minimize the size of the sensor 700 so as to be minimally invasive, it may not be practical to record all of the sounds picked up by the sensor. Therefore, the recording device 720 may include a buffer—such as a 5-30 second buffer—allowing the detected sounds to be reviewed and then store only those sounds meeting a predetermined criteria. It is contemplated that this determination will be made by the configurable signal processor 730. For example, the configurable signal processor 730 will analyze the sounds collected by the recording device 720. If a sound meets the criteria then that recording will be moved from the buffer into permanent storage in the memory unit 740 for later retrieval by an external unit. If a sound does not meet the criteria, then it will simply be ignored and the recording process will continue.

Recordings stored in the memory unit 740 may later be removed by an external device. As with other embodiments, it is contemplated that the external device will communicate with the sensor 700 via the telemetry unit 750. Once the external device has obtained the recordings from the memory unit 740 via the telemetry unit 750, then the recordings may either be played by the itself or transferred to another external unit adapted for playing the recordings, such as a speaker or other sound producing unit. In this manner the patient's doctor or a specialist may review the recorded for indications of changing tissue density or the onset of osteolytic lesions and choose a treatment plan accordingly. Similarly, the recordings may be analyzed using spectral analysis. Spectral analysis may include such analyzing techniques as Fast Fourier Transform algorithms, fuzzy logic, artificial intelligence, or any other method of analyzing the data. Utilizing spectral analysis may identify patterns in the sounds or detect problems that a general doctor or even a specialist might miss in reviewing the recordings. On the other hand, spectral analysis may provide a vehicle for allowing the doctor or specialist to better identify problems by converting the data into various visual forms such as spectrograms or other graphical representations.

It is also contemplated that the sound recordings may be analyzed with respect to each other over time. That is, the sound recordings do not have to be individually analyzed to determine changes in tissue density. Rather, comparing sound recordings over time may provide indications of tissue density changes. As previously mentioned, it is contemplated that in the case of utilizing the sensor in conjunction with an area having an artificial implant the sound recordings will change as the implant is initially integrated, then fully integrated, and then degrades. Thus, comparing sound recordings over time intervals may provide insight into tissue density changes and the potential for osteolytic lesion development. In this regard, it is fully contemplated that the sensor may be configured to allow recording of raw sound data by an external device. That is, the sensor need not include signal processing and memory. Rather, the sensor may simply facilitate the recording of sound data by a separate device. This sound data may be gathered at a plurality of sessions and then the data from the sessions compared by manual or computational means. This comparison will determine tissue conditions or changes in tissue.

It is not necessary for the sensor 700 to include a buffer. For example, the sensor 700 may have a memory unit 740 adapted for storing a certain amount of recordings from the recording device 720 such as hours, days, weeks, or months worth of recordings, or in terms of memory usage a certain number of bytes. Using such an approach, the data may be removed from memory unit 740 by an external device on an interval corresponding to the storage capacity of the memory unit. Thus, if the sensor 700 is configured for storing 30 hours worth of recordings on the memory unit 740, then a daily synchronization with the external device that removes and stores the recordings may be appropriate. Also this approach may obviate the need for including the signal processor 730 within the sensor 700. This is because, if all of the sounds observed by the transducer 710 are being recorded by the recording device 720, then the signal processing may be accomplished externally, either by the external device used to extract the data or another device, such as a computer, that may obtain the data from the external device and perform the signal processing.

If the sensor 700 does include a buffer and the signal processing is accomplished within the sensor, then it may be advantageous to also include a configurable signal processor 730. The configurable signal processor 730 is utilized as described above to discriminate between sounds satisfying a predetermined criteria and those that do not. The configurable signal processor 730 is also adapted for being configured by the external device. In this regard, the configurable signal processor 730 may communicate with the external device either via the telemetry circuit 750 or through a separate communication path. Either way, the external device may set, restore, or change such aspects of the configurable signal processor 730 as the predetermined criteria for keeping sound recordings, the type of tissue density data to be kept, the preset thresholds for normal tissue density signals, or any other setting related to the performance of the signal processor. Thus, a doctor can adjust the monitoring standards for the patient as conditions or available information changes. For example, as medical research continues to develop in this area and more is known of the specific sounds and signals indicative of different types of changes in tissue density, the sensor 700 may be adjusted via the configurable signal processor 730 to take such things into account and store the desired data accordingly.

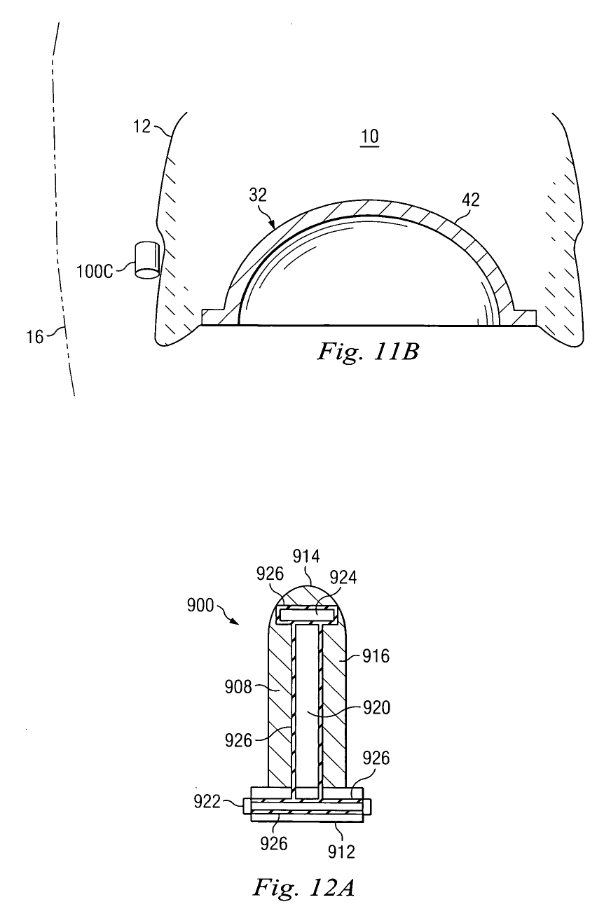

FIGS. 11A-11B illustrate a possible means of implanting a sensor 800 according to the present invention. The sensor 800 may be substantially similar to sensors 100, 300, 400, 500, 600, and 700 disclosed above. As shown in FIG. 10A and previously described, the sensor 800 may be shaped for implantation via a catheter 60. Without limitation, it is contemplated that the sensor 800 may take the shape of an elongated cylinder to facilitate placement via the catheter 60. In one aspect, the diameter of the sensor 800 is smaller than 10 mm. In another aspect, the diameter may be 4 mm or smaller. In another aspect, the diameter is smaller than 3 mm. The catheter 60 includes a proximal portion 62 adapted for being disposed outside of the patient's skin 16 and a distal portion 64 adapted for being disposed adjacent the implantation site 18 for the sensor 800. Sensor 800 may be positioned within the proximal portion 62 of the catheter 60 and then moved to the implantation site 18 by shaft 66. Shaft 66 is adapted to force the sensor 800 through the catheter 60 to the implantation site 18. The distal portion 64 of the catheter 60 may be shaped for accurate placement of the sensor 800.

FIG. 11B shows the sensor 800 disposed adjacent to the exterior bone surface 12 of bone 10. However, as with all the sensors of the present invention, it is contemplated that sensor 800 may be disposed adjacent a the tissue to be monitored, within the tissue, near the tissue, or distal to the tissue. Depending on the indicators being detected by the sensor 800, it is contemplated that the sensor may be located anywhere from a millimeter to several inches away from the exterior bone surface when disposed near the tissue. When the sensor 800 is disposed distal to the tissue being monitored, it is contemplated that the sensor may be several inches to several feet away from the tissue.

Sensor 800 has an external surface configured to engage the surrounding tissue to maintain its relative position in the body. Although sensor 800 is shown for the purposes of illustration as a cylinder, it will be appreciated that the outer surface of the body of the sensor 800, as well as any of the preceding sensors, may be shaped, to include tissue anchoring surfaces, or otherwise configured for maintaining the relative position of the implant with respect to the adjacent tissue. For example and without limitation, the outer surface may be threaded, knurled, ribbed, roughened, etched, sintered, bristled, have an ingrowth surface, or include protrusions to engage the surrounding tissue. Additionally, separately, or in combination with the foregoing, the outer surface may be at least partially coated with chemical or biologic agents for promoting adhesion to the adjacent tissue and/or growth of the tissue onto the outer surface of the sensor.

FIGS. 12A-13B show a sensor 900 according to one embodiment of the present invention that utilizes impedance as an indicator of changes in tissue density. Sensor 900 may be substantially similar to other embodiments of the present invention. Sensor 900 includes a main body 908. A head 912 of the sensor 900 includes a flange portion 918. A leading end 914 of the sensor 900 is adapted for being disposed within bone. To facilitate bone engagement the sensor 900 includes threads 916. The threads 916 are configured such that the sensor 900 may act as a bone screw. The sensor 900 also includes housing 920. The housing 920 is adapted for storing the electronics of the sensor 900, such as the integrated circuit, battery, data processor, memory, and communication devices. The housing 920 is insulated from any metal material of the main body 908, head 912, and leading end 914 by an insulator 926 to protect the electronics and allow the sensor 900 to function properly. The electronics are connected to electrodes 922 and 924. It is contemplated that electrodes 922 and 924 may be ring, band, or any other type of electrode capable of measuring impedance. Electrodes 922 and 924 are also insulated from any metal material of the main body 908, head 912, and leading end 914 of the sensor 900 by insulator 926. The sensor 900 and its electronics are adapted for measuring the impedance between electrodes 922 and 924.

It is contemplated that the electrodes 922 and 924 may be located completely within the main body 908, head 912, and leading end 914 of the sensor. However, as shown in FIG. 12A it is also contemplated that the electrode 922 may extend beyond the boundaries of the head 912. In this respect, the electrode 922 may be adapted to contact a metal portion of an implant so as to cause the entire metal portion of the implant to act as an electrode. In that case, the impedance would be measured between electrode 924 and the metal portion of the implant, providing a wider range of detection for tissue density changes. Electrode 924 may be similarly configured to contact an implant to cause the implant to act as an electrode.

FIG. 12B shows the sensor 900 implanted and engaged with a portion of an implanted portion of a hip prosthesis, the acetabular cup 32, so as to cause the implant to act as an electrode. Once the sensor 900 is in place the electrode 922 will be in contact with the metal acetabular cup 32. Impedance will then be measure between electrode 924 and exterior surface 42 of the acetabular cup 32. Changes in tissue density, including bone degradation, are monitored by the electric impedance measurement between electrode 924 and exterior surface 42. As in other embodiments, it is contemplated that sensor 900 may store the impedance data for later retrieval or may simply immediately communicate the impedance data to an external device. It is also contemplated that a plurality of impedance sensors may be used. In that case, impedance may be measured not only between the electrodes of each sensor, but also between sensors as well. This can further expand the region of tissue that is monitored for changes in density.

FIGS. 13A and 13B show an alternative embodiment of the present invention sensor 900A. Sensor 900A is substantially similar to sensor 900 described above. However, in this embodiment electrode 922 of sensor 900A is insulated from the acetabular cup 32 as well as the metal portions of the sensor itself, but is exposed to the space underneath inner surface 40 where the ball-in-socket motion of the artificial hip joint occurs. The fluidic environment of this space contributes to the electric impedance between electrodes 922 and 924. The ball-in-socket motion of the hip joint will modulate the electric impedance between electrodes 922 and 924. In one embodiment this modulated signal can be used as a pedometer to track use of the implant. Further, surrounding tissue inflammation can contribute to acidic fluid within the space. The acidic fluid can increase the electric conductance and the corresponding change in impedance can indicate inflammation in the tissue, which is often an indication of changes in tissue density such as the onset of osteolysis.

The various embodiments of the present invention may have particularly useful application in tracking the healing of tissue, including the rate of healing and effectiveness of treatments. For example, the sensors may be adapted to be implanted into or adjacent the spine to detect indicators of improving bone quality in fusion and grafting procedures. In a spinal interbody fusion, the sensor may be utilized to determine more accurately when the vertebrae have fully fused together. In one embodiment, micro-motion sounds associated with unfused bone may be used to determine when sufficient fusion has occurred. Alternatively, the changes in conductivity energy (e.g. acoustic or electric) may be sensed to determine the degree of bone fusion. Similar techniques may be used in the case of ankle and other bone fusions as well. Similarly, in the case of dental implants requiring implantation of a post into the alveolar ridge it is common to wait six months to allow the allograft, autograft, synthetic bone, or other material to be incorporated into the jaw before implanting the post. However, utilizing the current invention the sensor can use indicators of bone density or a determined rate of healing to predict when the graft is fully healed without waiting for a very conservative length of time to pass.

The sensors may also be used to monitor treatment of a tissue. For example, in the treatment of osteoporosis it is common to give the patient vitamin D, calcium supplements, bisphosphonates, or other pharmaceuticals and then monitor the patient's bone mineral density. Sensors according the present invention provide a way to monitor changes, both good and bad, in bone mineral density and help facilitate treatment of osteoporosis. The sensors may be particularly advantageous in treating osteoporosis in the areas around artificial implants where the implant interferes with the ability to use dual energy x-ray absorptiometry to determine bone mineral density.

The sensors may also be used to control bone growth stimulators. That is, it is contemplated that the sensors may be used in combination with bone growth stimulators—chemical, electrical, biological, or otherwise—to determine a course of treatment. For example, the sensors may be utilized to determine when there has been sufficient bone growth to halt the use of the bone growth stimulator. On the other hand, the sensors may also be used to detect slowing in bone growth and the need to increase the amount of bone growth stimulation. It is fully contemplated that the sensors may be in communication with a bone growth stimulator release mechanism so that the proper amount of bone growth stimulation is provided based on the sensors' determinations. The parameters for the sensors' determinations may be programmed into the sensor based on the treating physician's preference. As described previously, it is contemplated that the sensor may be programmable so that the treating physician may change the parameters for the sensor after implantation to facilitate changes in the treatment of the tissue and, in particular, the amount of bone growth stimulation.

As briefly described previously, it is contemplated that the sensors according the present invention may utilize a variety of alternative techniques to power the sensor. For example, it is fully contemplated that the sensor may be piezoelectric. It is also contemplated that the sensor may simply use the kinematics of the body for power. Further, though the sensors described above have mostly been described as passive in the sense that they listen for indicators created by the body itself, it is also contemplated that the sensor may be powered such that it can send out a signal. Under such an approach, the sensor may utilize pulse-echo type sensing. The sensor would send out a signal and then listen for the echo. Based on the echo the sensor could then detect changes in tissue density. Similarly, instead of a pulse-echo system, a signal generator and a sensor could be utilized. The signal generator would send out a signal and the sensor would receive the signal and based on changes in the detected signals indicate changes in tissue density. When detecting an emitted signal, either in pulse-echo or generator-sensor mode, it is contemplated that the signal may be acoustic, electric, or any other type of transmission that may be utilized to detect changes in tissue density.

While the foregoing description has been made in reference to hip, knee, spine, ankle, and jaw joints, it is contemplated that the disclosed sensor may have further applications throughout the body. Specifically, such disclosed sensors may be useful to evaluate tissue density and detect changes to tissue throughout the body. It is contemplated that the sensor may have particular application with respect to detecting changes in bone density as it relates to osteoporosis. Further, the sensor may be applied to detect tissue density changes with respect to tissue around fixation implants, joint implants, or any other type of implant. The sensor may also be applied to detect disc bulges or tears of the annulus when applied in the spinal region. Moreover, an acoustic sensor may also be used to detect changes in viscosity. Thus, the sensor may be utilized to listen for changes in bodily systems and organs and alert healthcare professionals to any impending or potential problems. Further, the sensor may be used in cooperation and/or communication with an implanted treatment device such as a pump or a stimulator. The pump or stimulator may be controlled based on the readings sensed by the sensor. These examples of potential uses for the sensor are for example only and in no way limit the ways in which the current invention may be utilized.

Further, while the foregoing description has often described the external device as the means for displaying sensor data in human intelligible form, it is fully contemplated that the sensor itself may include components designed to display the data in a human intelligible form. For example, it is fully contemplated that the sensor may include a portion disposed subdermally that emits a visible signal for certain applications. Under one approach, the sensor might display a visible signal when it detects indicators indicative of an osteolytic lesion. The sensor might also emit an audible sound in response to such indicators. In this sense, the sensor might act as an alarm mechanism for not only detecting potential problems but also alerting the patient and doctor to the potential problems. This can facilitate the early detection of problems. Under another approach, the sensor might display a different color visible signal depending on the indicators detected. For example, but without limitation, in the case of measuring tissue density the sensor might emit a greenish light if the indicators detected by the signal indicate density is within the normal range, a yellowish light if in a borderline range, or a red light if in a problematic range.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

What is claimed is:

1. An implantable sensor for detecting indicators of the density of a tissue in a body, comprising:
a sensor having an external surface adapted to engage a portion of the body to maintain a position in the body, the sensor configured for detecting a signal indicative of a density of the tissue; and
a telemetry circuit in communication with the sensing element adapted for transmitting the detected signal outside of the tissue.

2. The implantable sensor of claim 1, wherein the sensing element is adapted for detecting acoustic signals.

3. The implantable sensor of claim 1, wherein the sensing element is adapted for detecting impedance signals.

4. The implantable sensor of claim 1, wherein the sensor is externally powered.

5. The implantable sensor of claim 4, wherein the telemetry circuit is adapted for transferring power from an external device to the sensing element.

6. The implantable sensor of claim 5, wherein the telemetry circuit includes a coil adapted for inductive coupling.

7. The implantable sensor of claim 1, wherein the portion of the body engaged by the external surface is the tissue.

8. The implantable sensor of claim 1, wherein the sensor is internally powered.

9. The implantable sensor of claim 1, wherein the tissue is a bone.

10. The implantable sensor of claim 9, wherein the sensor is adapted for detecting indicators of an osteolytic lesion.

11. The implantable sensor of claim 1, wherein the tissue is a soft tissue.

12. The implantable sensor of claim 1, wherein the portion of the body engaged by the external surface is a bone.

13. The implantable sensor of claim 1, wherein the portion of the body engaged by the external surface is a soft tissue.

14. The implantable sensor of claim 1, wherein the portion of the body engaged by the external surface is adjacent an artificial implant.

15. A system for detecting changes in tissue density, comprising:
an implantable acoustic sensor adapted for detecting a signal indicative of a density of a tissue and communicating the signal to an external receiver; and
an external receiver adapted for receiving the signal from the implantable sensor.

16. The system of claim 15, wherein the sensing element is adapted for detecting sounds.

17. The system of claim 15, wherein the sensing element is adapted for detecting vibrations.

18. The system of claim 15, wherein the implantable sensor is externally powered.

19. The system of claim 15, wherein the external receiver is adapted for providing power to the implantable sensor.

20. The system of claim 15, wherein the implantable sensor is internally powered.

21. The system of claim 20, wherein the implantable sensor includes a battery.

22. The system of claim 20, wherein the implantable sensor includes a memory unit adapted for storing tissue density data representative of the detected signals.

23. The system of claim 22, wherein the implantable sensor includes a signal processor.

24. The system of claim 23, wherein the tissue density is the density of a portion of a bone.

25. The system of claim 24, wherein the signal processor is adapted for classifying signals that are indicators of an osteolytic lesion.

26. The system of claim 25, wherein the memory unit is adapted for storing data corresponding to the osteolytic lesion indicators.

27. The system of claim 15, wherein the external receiver includes a signal processing unit adapted for creating tissue density data representative of the signals received from the implantable sensor.

28. The system of claim 27, wherein the external receiver includes a memory unit adapted for storing the tissue density data.

29. The system of claim 27, wherein the external receiver includes an output mechanism.

30. The system of claim 28, wherein the output mechanism is configured for outputting the tissue density data in a human intelligible form.

31. The system of claim 30, wherein the human intelligible form is a visual display.

32. The system of claim 29, wherein the output mechanism is configured for sending the tissue density data over a network.

33. The system of claim 15, wherein communication between the implantable sensor and the external receiver is wireless.

34. The system of claim 33, wherein the wireless communication is RFID communication.

35. The system of claim 15, further comprising a plurality of implantable acoustic sensors.

36. The system of claim 35, wherein the plurality of implantable acoustic sensors operate as redundancies.

37. The system of claim 35, wherein the plurality of implantable acoustic sensors operate together.

38. The system of claim 37, wherein the plurality of implantable acoustic sensors are adapted to locate a tissue density change based on the detected signals.

39. The system of claim 15, wherein the implantable sensor is adapted for percutaneous implantation.

40. The system of claim 39, wherein the implantable sensor is substantially cylindrical.

41. The system of claim 40, wherein the implantable sensor has a diameter less than 10 mm.

42. The system of claim 41, wherein the implantable sensor has a diameter less than 4 mm.

43. The system of claim 39, wherein the external receiver is implantable.

44. The system of claim 43, wherein the external receiver is adapted for percutaneous implantation.

45. A method of detecting a density of a tissue in a body, comprising:
providing a sensor adapted for detecting a signal indicative of a density of a tissue, the sensor having an external configuration adapted to engage a portion of the body to maintain a position in the body;
inserting the sensor into the body adjacent a site to be monitored;
engaging the external configuration with a portion of the body to maintain the position of the sensor; and
operating the sensor to detect a signal indicative of a density of the site.

46. The method of claim 45, wherein the sensor is an acoustic sensor.

47. The method of claim 45, wherein the sensor is an impedance sensor.

48. The method of claim 45, further comprising operating the sensor to detect a plurality of signals indicative of the density of the site.

49. The method of claim 48, further comprising analyzing the signals with respect to one another to detect changes in density.

50. The method of claim 49, wherein the analyzing includes comparison of a first audible signal with a second audible signal.

51. The method of claim 49, wherein the analyzing includes spectral analysis.

52. The method of claim 45, wherein the inserting includes percutaneously positioning the sensor.

53. The method of claim 52, wherein the inserting includes passing the sensor through a catheter.

54. The method of claim 45, wherein the site is an interface between an artificial implant and natural tissue.

55. The method of claim 54, further including implanting an artificial implant after inserting the sensor.

56. The method of claim 54, further including implanting an artificial implant prior to inserting the sensor.
